(19) United States
(12) Patent Application Publication
(10) Pub. No.: US 2020/0281682 A1
(43) Pub. Date: Sep. 10, 2020

(54) SHARPS COLLECTION TRAY AND CONTAINER SYSTEM

(71) Applicant: Daniels Family Investment Holdings Pty, Ltd, Dandenong (AU)

(72) Inventors: Christopher Alexander Hart, Preston (AU); Hubertus Pennings, Ferntree Gully (AU)

(21) Appl. No.: 16/295,172

(22) Filed: Mar. 7, 2019

(51) Int. Cl. A61B 50/36; A61B 50/33

(52) U.S. Cl. CPC A61B 50/362; A61B 50/3001; A61B 50/33

(57) ABSTRACT

A sharps collection system includes a container and a tray. The tray conveys medical waste from a drop area to a storage area in the container. The tray includes a proximal-most edge, a distal-most edge opposite the proximal-most edge, and an acceleration zone between the proximal-most edge and the distal-most edge. in addition, the tray includes one or more features that perform one or more of the following functions: (1) accelerate movement of waste out of the drop area and into the storage area, (2) minimize the effects of adverse conditions, such as moisture or sticky surfaces on the tray that impede or prevent movement of waste, and (3) expedite the return of the tray from a drop position to an equilibrium position to receive waste more readily.

### FIELD

The present invention relates generally to systems for the collection and disposal of medical waste, and more particularly to container systems with trays that more rapidly convey discarded items to a secure storage area.

### BACKGROUND

Needle syringes, scalpel blades, and other medical “sharps” can carry dangerous blood borne pathogens after they are used. Medical professionals can become exposed to these pathogens through accidental contact with contaminated sharps. Therefore, it is important that contaminated sharps be collected and removed from work areas as soon as is possible after use.

Numerous types of containers have been developed for the purpose of collecting contaminated sharps devices and storing them in an area where the sharps are no longer accessible to personnel. For example, some sharps disposal containers include a storage receptacle with some type of lid or door. The user can pivot open the lid or door and deposit contaminated sharps into the receptacle. In some instances, the container may have a pivoting tray that receives the contaminated sharps. The tray may be designed to catch a contaminated syringe that a user drops into the container, at which point, the weight of the syringe causes the tray to pivot from its initial orientation (or “receiving position”) to a more vertical orientation (or “drop position”). As the tray pivots to a drop position, the syringe can fall into the receptacle by gravity.

The above-described container systems generally operate on the same two step principle. First, a contaminated sharps is dropped into an area of the container (hereinafter referred to as the “drop area”) that is accessible to, and directly reachable with, a user's hand. Second, the contaminated sharps is conveyed from the drop area to another area of the container (hereinafter referred to as the “storage area”) that is inaccessible to the user.

Although conventional sharps containers provide an adequate solution for storing and disposing of medical wastes, they still have significant drawbacks. In particular, there are many issues that can occur in the drop area that are not accounted for, and in some cases, are completely overlooked by conventional container and tray designs. Designers of conventional containers often assume, incorrectly, that items dropped onto a tray will be safely removed from a drop area by gravity. Gravity alone does not ensure that contaminated sharps will move quickly and safely out of the drop area, however. Many variables can compete with gravity to prevent sharps from quickly leaving the drop area and entering the storage area. For example, lighter items that are dropped onto conventional trays may not be heavy enough to “activate” or tilt the tray from the receiving position to the drop position. In such instances, the light-weight item will not readily roll or slide off of the tray into the storage area. This lack of responsiveness of the to tray can keep lighter items in the drop area longer than desired, increasing the potential that a user will come into accidental contact with the item in the drop area.

Items dropped into conventional containers are also prone to “bounce back”. Bounce back can occur when an item is dropped onto the tray from an elevation above the tray. In such instances, the item can bounce upwardly off of the tray surface. This is bounce back creates a moment in time when the item is airborne and not safely received in the drop area. During this moment in time, the item's momentum is directed upwardly and away from the tray, rather than toward a rolling momentum down the tray. As a result, the item develops less momentum to propel it down the tray, and therefore takes longer to leave the drop area. This additional time that the item is in the drop area delays the passage of the item into the secure storage area, and therefore increases the potential that a user can come into harmful contact with the item.

Conventional trays can also lack mechanisms for addressing blood, liquids, and residues that accumulate on the surface of the tray. These residues can cause items to adhere to the tray, rather than fall into the storage area, even after the tray pivots to the drop position. Adherence is a particular problem for flat items like scalpel blades. When a flat item like a scalpel blade is dropped onto a flat wet tray surface, adhesion forces can develop between the liquid and the blade that cause the blade to stick to the surface of the tray, rather than slide off the tray by gravity. Liquids like blood have relatively high capillary action that can cause the liquid to spread beneath the entire blade and increase adhesion.

Finally, conventional trays can take longer than desired to return from the drop position back to the receiving position. Many trays are designed to receive items only when they are in the receiving position, and not admit items into the container when they are in the drop position. Therefore, it is desirable for a tray to return to the receiving position as quickly as possible after it tilts to a drop position, so that the tray is ready to receive another item. Even a short delay in returning to the receiving position can prolong the handling of contaminated sharps and increase the potential for needle sticks, puncture wounds and other types of accidental contact.

These and other shortcomings of known sharps collection systems can delay or prevent removal of contaminated sharps out of the drop area. When contaminated sharps are not moved quickly out of the drop area, there is more potential for an unsuspecting user not looking at the drop area to place their hand in the drop area while dropping an item, and come in contact with an item(s) previously deposited into the drop area. This can result in contact with contaminated item(s) that are still in the drop area, exposing the user to blood borne pathogens. Therefore, conventional sharps collection systems have areas in need of significant improvement, Including areas in and around the drop area.

### SUMMARY

The drawbacks of conventional sharps collection systems are resolved in many respects by sharps collector trays and containers in accordance with the present disclosure.

In one beneficial aspect of this disclosure, a tray is used for conveying medical waste from a drop area to a storage area of a sharps collection container. The tray includes a proximal-most edge, a distal-most edge opposite the proximal-most edge, and an acceleration zone between the proximal-most edge and the distal-most edge. The tray defines a tray axis that extends from the proximal-most edge to the distal-most edge. The acceleration zone includes a base surface and elongated rails projecting above the base surface. The elongated rails collectively form a conveyance surface above the base surface for transporting medical waste toward the distal-most edge while limiting or preventing contact between the medical waste and the base surface.

In another beneficial aspect of this disclosure, a tray for conveying medical waste includes an arcuate landing adjacent the proximal-most end of the tray.

In another beneficial aspect of this disclosure, a tray for conveying medical waste includes an arcuate landing having a radius of curvature that increases as the arcuate landing extends toward the distal-most end.

In another beneficial aspect of this disclosure, a tray for conveying medical waste includes a ramp that terminates at the distal-most end of the tray.

In another beneficial aspect of this disclosure, a tray for conveying medical waste includes a ramp that is inclined upwardly relative to the acceleration zone.

In another beneficial aspect of this disclosure, a tray for conveying medical waste includes rails that extend parallel to one another.

In another beneficial aspect of this disclosure, a tray for conveying medical waste includes rails, each rail having a first rail wall and a second rail wall opposite the first rail wall.

In another beneficial aspect of this disclosure, a tray for conveying medical waste includes rails, each rail having a first rail wall and second rail wall that extend parallel to to the tray axis.

In another beneficial aspect of this disclosure, a tray for conveying medical waste Includes first rail walls, second rail walls, and a base surface that define channels for conveying liquid toward the distal-most end by channel flow.

In another beneficial aspect of this disclosure, a tray for conveying medical waste is includes channels, wherein each channel has a channel width extending transversely to the tray axis, the channel width being between about ⅛ inch to about 1 inch.

In another beneficial aspect of this disclosure, a tray for conveying medical waste includes rails with top surfaces raised above the base surface, the top surfaces collectively forming a conveyance surface for conveying waste into a storage area.

In another beneficial aspect of this disclosure, a tray for conveying medical waste includes rails with top surfaces that are rounded.

In another beneficial aspect of this disclosure, a tray for conveying medical waste includes a first side wall and a second side wall, the first and second side walls projecting above the base surface and above elongated rails on the tray.

In another beneficial aspect of this disclosure, a tray for conveying medical waste includes a first side wall and a second side wall, wherein the first side wall and the second side wall each include a pivot mount for pivotally mounting the tray inside a sharps collection system.

In another beneficial aspect of this disclosure, a sharps collection system includes a tray having any of the aforementioned features.

### BRIEF DESCRIPTION OF THE DRAWING FIGURES

The present disclosure will be better understood with reference to the non-limiting examples shown in the accompanying drawing figures, of which:

FIG. 1 is a perspective view of a sharps collection system in accordance with one example, the sharps collection system shown in a first operating condition;

FIG. 2 is a perspective view of the sharps collection system in FIG. 1, the sharps collection system shown in a second operating condition;

FIG. 3 is a truncated side view of the sharps collection system in FIG. 1, with an item being dropped into the sharps collection system;

FIG. 4 is a truncated perspective view of the sharps collection system of FIG. 3, with the Item rolling toward a distal-most edge of a tray inside the sharps collection system;

FIG. 5 is a perspective view of a tray in accordance with one example of trays used in sharps collection systems of the present disclosure;

FIG. 5A is an enlarged view of an area of the tray shown in FIG. 5;

FIG. 6 is a perspective view of a tray in accordance with another example of trays used in sharps collection systems of the present disclosure;

FIG. 7 is truncated side section view of a tray and cowl used in the sharps collection system of FIG. 1;

FIG. 7A is an enlarged side section view of an area of the tray shown in FIG. 7; and

FIG. 8 is a schematic side view of the tray and cowl in FIG. 7, showing different pivot positions of the tray.

### DETAILED DESCRIPTION

Although this disclosure contains descriptions of specific embodiments, the disclosure is not intended to be limited to the details shown. Rather, various modifications to the details described herein, and/or various combinations of such details, may be made without departing from the disclosure. This disclosure is best understood from the following detailed description when read in connection with the accompanying drawing figures, which show exemplary embodiments that are selected for illustrative purposes. The accompanying figures are intended to be illustrative rather than limiting, and are included herewith to facilitate the explanation of the embodiments.

Sharps container systems in accordance with this disclosure address the problem of moving contaminated sharps from the drop area of the container system to the storage area of the container system as quickly and efficiently as possible. This is accomplished through one or more of the following features: (1) features that accelerate the movement of waste out of the drop area, (2) features that minimize the occurrence and effects of adverse conditions that impede movement of waste, and (3) features that expedite the return of the tray from a drop position to the receiving position to receive waste more readily.

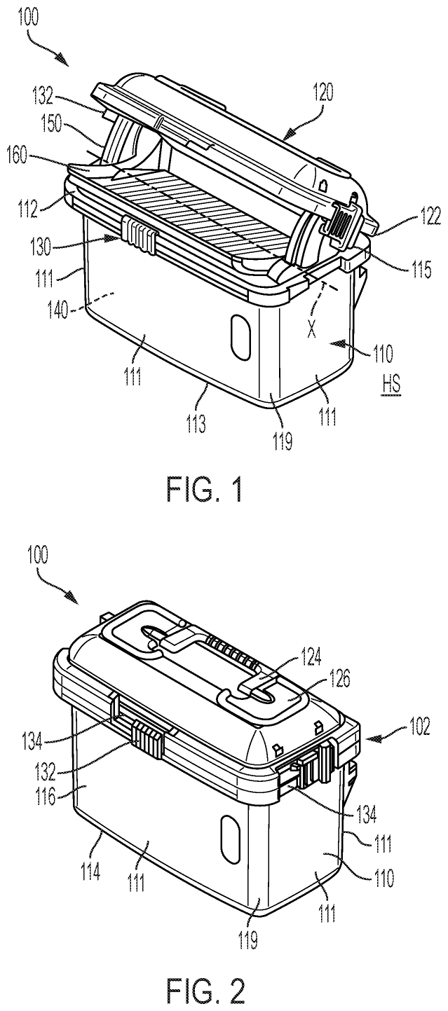

Referring now to FIGS. 1 and 2, a sharps collection system or “container” 100 is shown in accordance with one exemplary embodiment. Container 100 is a generally rectangular enclosure 102 that receives and stores medical wastes for subsequent transportation and disposal. Enclosure 102 has a bottom portion in the form of a receptacle 110. Receptacle 110 has four side walls 111 arranged in a generally rectangular shape, with each side wall intersecting along a generally vertical corner edge 119 that is rounded. Each side wall has a top edge 112 and a bottom edge 113. Bottom edges 113 are joined together by a bottom wall 114. Top edges 112 surround a top opening 115. In this arrangement, side walls 111 and bottom wall 114 form a partial enclosure 116 that defines a storage area 140 for receiving medical waste.

Container 100 also includes a top portion in the form of a door or lid 120. Lid 120 is pivotally attached to receptacle 110 on one side of the receptacle with a hinged connection 122. Hinged connection 122 is configured to permit lid 120 to pivot relative to receptacle 110 between an open position (FIG. 1) and a closed position (FIG. 2). Receptacle 110 and lid 120 have security measures in the form of security locks 130 on multiple sides. Each lock 130 features a first lock member 132 and a second lock member 134. Two of the first lock members 132 are attached to lid 120, and one of the first lock members is attached to receptacle 110. Likewise, two of the second lock members 134 are present on receptacle 110, and one of the second lock members is present on lid 120. Each first lock member 132 is slidable between two positions or modes when lid 120 is in the closed position: (1) a locked position to lock the lid in the closed position, and (2) an unlocked position to allow the lid to be moved from the closed position to the open position. In the locked position, first lock member 132 is advanced over second lock member 134 to cover the second lock member and block the lid from moving to the open position. In the unlocked position, first lock member 132 is moved away from second lock member 134 to expose the second lock member and allow the lid to move to the open position.

It will be appreciated that containers in accordance with this disclosure can have various shapes and dimensions. In addition, containers in accordance with this disclosure can be provided with different storage capacities. Moreover, containers in accordance with this disclosure can be configured as single-use containers that are disposable with the medical waste inside them, or as reusable containers that can be emptied of waste, sterilized and reprocessed. In the present example, container 100 is designed as a portable single-use container that can be carried by hand when the container is empty or full. Lid 120 has a handle 124 attached to a top surface 126. When lid 120 is in the closed position, and first lock members 132 are in the locked positions, a user can manually grip handle 124 to lift and carry container 100.

It will be further appreciated that containers in accordance with the invention can features shapes, dimensions, and storage capacities that are different than that of container 100, in which case, other configurations for receptacles, lids, trays, handles and locks can be used without departing from the scope of this disclosure.

Receptacle 110 and lid 120 collectively define a drop area 150 between them for receiving medical waste when the lid is in the open position. A tray 160 is mounted inside receptacle 110 and movable between two positions as lid 120 is moved between the open and closed positions. When lid 120 is moved to the open position, tray 160 assumes a generally horizontal receiving position, in which the tray partially projects outwardly and above top edge 112 of receptacle 110, as shown in FIG. 1. In the receiving position, tray 160 is oriented to catch medical waste deposited into drop area 150. When lid 120 is moved to the closed position, as shown in FIG. 2, tray 160 is pivoted and lowered into container 100 in a “stowed position”.

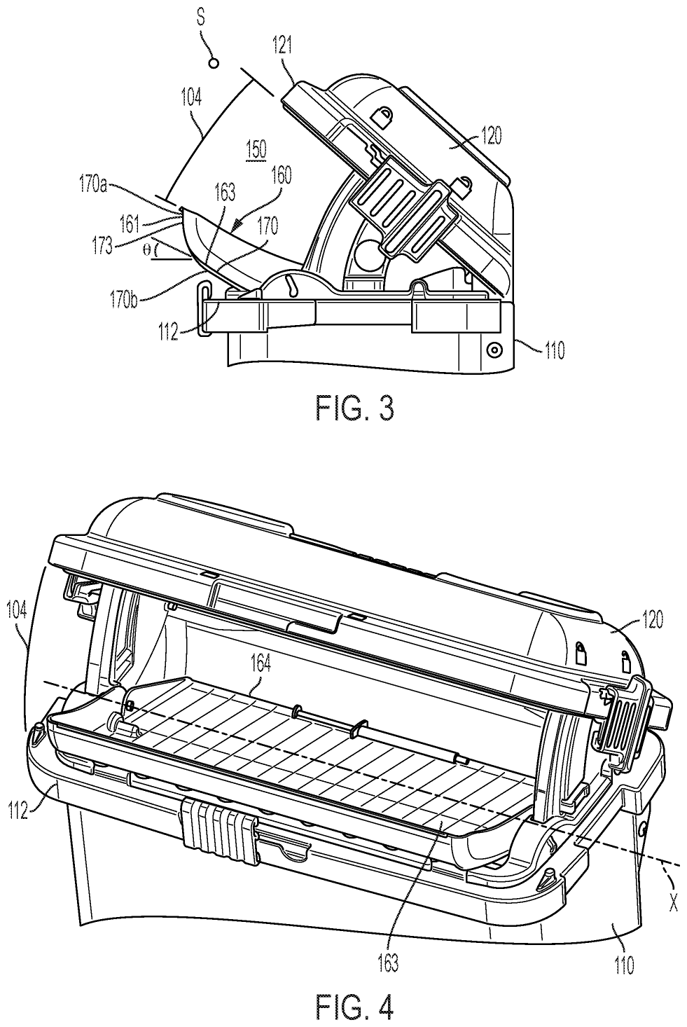

FIGS. 3 and 4 provide additional views of container 100 with lid 120 moved to the open position, and with tray 160 moved to the receiving position. In this mode, a front portion 161 of tray 160 projects in a forward direction (defined in this context as toward the left side of FIG. 3) relative to a front edge 121 of lid 120. Front portion 161 of tray 160 also extends above top edge 112 of receptacle 110. In this arrangement, tray 160 and lid 120 collectively form a mouth 104 into drop area 150. Mouth 104 provides a portal through which medical waste can be deposited from outside of container 100 into drop area 150. Users of container 100 have direct access to drop area 150 through mouth 104. However, users of container 100 do not have direct access to storage area 140 through mouth 104, because tray 160 forms a barrier between the mouth and the storage area when lid 120 is in the open position.

Tray 160 is pivotally mounted in container 100 on a horizontal pivot axis X extending through a midportion of the tray. When container 100 is placed on a generally horizontal surface HS in an upright position (defined in this context as the position and orientation shown in FIG. 1), tray 160 assumes a position of equilibrium on pivot axis X (hereinafter, the “equilibrium position”) when lid 120 is in the open position. In the equilibrium position, tray 160 is in its receiving position and oriented so as to slope downwardly in a rearward direction (defined in this context as toward the right side of FIG. 3). This creates a downwardly sloping landing surface 163 that immediately moves medical waste out of drop area 150 and into an inner portion of container 100 after the medical waste lands on tray 160.

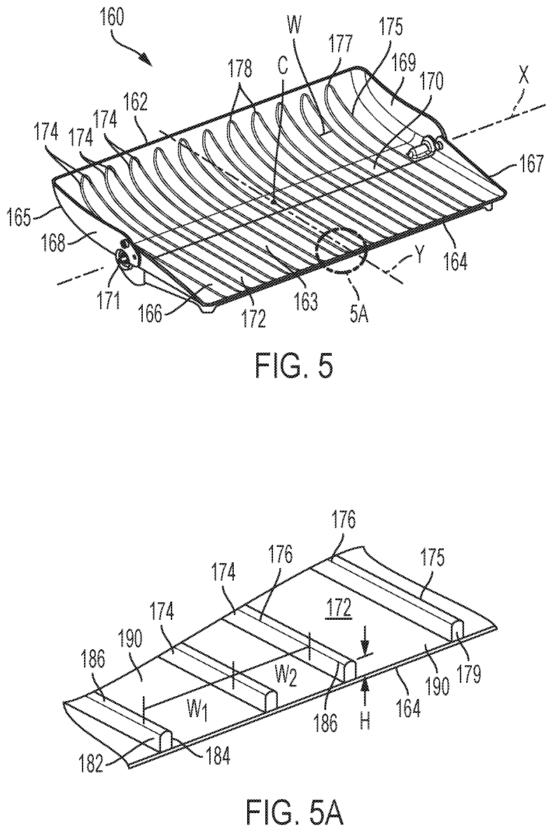

Referring to FIG. 5, tray 160 includes a proximal-most edge 162 and a distal-most edge 164 opposite the proximal-most edge. For purposes of this description, the term “proximal-most edge” is defined as the point or points on the tray that project the farthest toward the frontward direction, corresponding to the left side of FIG. 3. The term “distal-most edge” is defined as the point or points on the tray that project the farthest toward the rearward direction, corresponding to the right side of FIG. 3. Items that are dropped onto tray 160 are conveyed away from proximal-most edge 162 of the tray and toward distal-most edge 164 of the tray when tray 160 is mounted in container 100 in the equilibrium position.

Trays in accordance with this disclosure can have one or more features that increase the “activation response time” or “sensitivity” of the tray. The terms “activation response time” and “sensitivity” are both used herein to refer to the amount of time it takes for a tray to tilt out of the receiving position and toward a drop position, starting from when an item of a given weight first contacts the landing surface of the tray. Referring to FIG. 3 in the present example, acceleration zone 170 has a curved profile. When tray 160 is in the receiving position, acceleration zone 170 has an average angle of inclination θ of 30 degrees from horizontal. Angle of inclination θ is larger than known designs, creating a steeper descent that is more sensitive to lighter-weight items, and more quickly moves lighter items down tray 160.

Trays in accordance with this disclosure can also have one or more features that minimize the occurrence of bounce back when items are dropped onto the trays. In the present example, acceleration zone 170 has a curved transition 173 near proximal-most edge 162. Curved transition 173 connects a generally vertical section 170a of acceleration zone 170 to a more horizontal section 170b of the acceleration zone. In this arrangement, landing surface 163 gradually transitions dropped items from their initial trajectory, which is substantially vertical, to a more horizontal trajectory. Therefore, items do not immediately land on a generally horizontal planar surface, which can cause bounce back. Instead, the rounded contour of landing surface 163 causes items to transition smoothly to from a substantially vertical trajectory to a more horizontal trajectory. This causes the items to remain in contact with tray 160, rather than bounce, which promotes more rolling motion through acceleration zone 170. Rolling motion causes items to exit the tray more quickly than motion that is interrupted by bounce back.

Trays in accordance with the present disclosure can additionally include one or more structures on their periphery to direct items toward the distal-most edge of the tray, and prevent items from falling off of the landing surface before reaching the distal-most edge. Referring to FIG. 5, tray 160 includes a left lateral-most edge 165 and a right lateral-most edge 167 opposite the left lateral-most edge. Left lateral-most edge 165 includes a left sidewall 168, and right lateral-most edge 167 includes a right sidewall 169 opposite the left sidewall. Left sidewall 168 and right sidewall 169 project upwardly from landing surface 163, providing a barrier on each side of tray 160 that prevents items from rolling or sliding off the sides of the tray before reaching the distal-most edge.

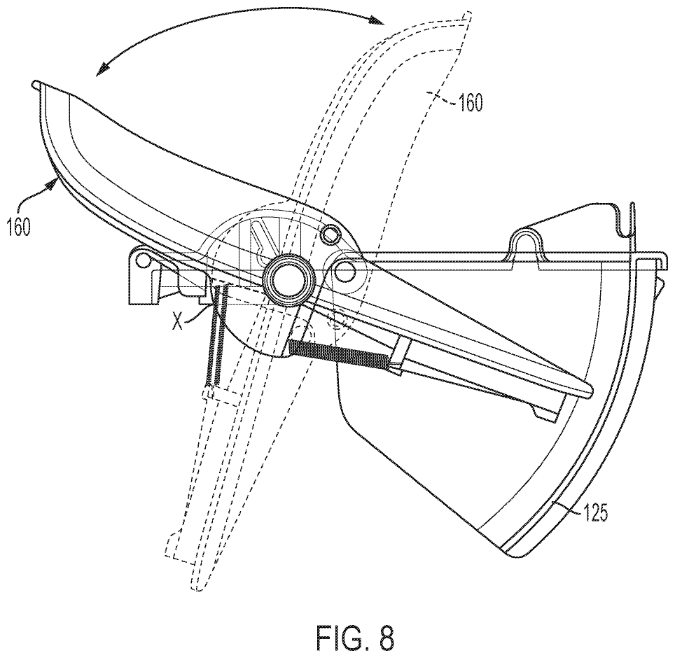

Left sidewall 168 and right sidewall 169 also include pivot mounts 171 for pivotally mounting tray 160 inside container 100. Pivot mounts 171 are configured to support tray 160 in a pivotal arrangement in which the tray pivots about axis X. In this arrangement, pivot mounts 171 allow tray 160 to pivot in response to an item or items being dropped onto landing surface 163. When tray 160 is empty, the center of gravity C of the tray is vertically aligned with pivot axis X, with landing surface 163 in a downwardly sloped orientation. After an item is dropped onto tray 160, the item rolls or slides along landing surface 163 and toward the distal-most edge 164 by gravity. After the item rolls or slides past a certain point of tray 160, the combined center of gravity of the tray and item will move closer to the distal-most edge 164 and away from pivot axis X. This shift causes tray 160 to pivot out of the equilibrium position and toward a drop position. When tray 160 reaches a drop position, the item will roll or slide off of distal-most edge 164 and fall into receptacle 110. FIG. 8 schematically shows tray 160 in solid line, representing the equilibrium position, and in dashed line, representing a pivoted position that the tray can reach after dropping an item into storage area 140. After the item falls off of tray 160 into receptacle 110, center of gravity C causes the tray to pivot back to the equilibrium position.

Some containers in accordance with the present disclosure may include safety features in addition to the tray to prevent users from accessing the interior of the receptacle after items are dropped into the container. In the present example, container 100 includes an arc-shaped cowl 125, as shown in FIGS. 7 and 8. Cowl 125 includes a rounded body 126 that extends beneath tray 160. In this position, tray 160 and cowl 125 extend between mouth 104 of container 100 and the interior of receptacle 110. Cowl 125 extends far enough below tray 160 so that there is never a clear and unobstructed passage between mouth 104 of container 100 and storage area 140, regardless of the tray's position. Rounded body 126 has an arc-shaped receiving surface 127 in proximity to distal-most edge 164 of tray 160. Receiving surface 127 is spaced from tray 160 to provide a clearance 129 between the tray and cowl 125 throughout a substantial portion of the tray's pivot range. Receiving surface 127 is configured to receive or “catch” items that roll or slide off of tray 160 and direct those items downwardly and beneath the tray.

Trays in accordance with the present disclosure include an acceleration zone that extends between the proximal-most edge and distal-most edge of the tray. Acceleration zones in accordance with the present disclosure can include one or more features configured to minimize or eliminate: (1) elements that impede the normal acceleration of items as the items move on the tray by gravitational force, and/or (2) elements that impede the return of the tray to the receiving position. These features can include, but are not limited to, features that reduce the effective surface area of the tray in contact with items, features that prevent accumulation of moisture on the tray, and features that increase the momentum of the tray as it pivots.

In the present example, tray 160 includes an acceleration zone 170 that extends between proximal-most edge 162 and distal-most edge 164. Acceleration zone 170 includes multiple features that minimize or eliminate elements that impede the normal acceleration of items on the tray being conveyed by gravitational force, and elements that impede the return of the tray to the receiving position.

Tray 160 defines a tray axis Y that extends from proximal-most edge 162 to distal-most edge 164, as shown in FIG. 5. Acceleration zone 170 includes a base surface 172 that extends on a top surface 166 of tray 160. Acceleration zone 170 also includes plurality of elongated rails 174 that project upwardly from base surface 172. The majority of surface area on top surface 166 is occupied by base surface 172. A small minority of surface area on top surface 166 is occupied by the rails 174. In this arrangement, rails 174 collectively form a conveyance surface 176 which extends above base surface 172, and which has a very small surface area as compared to the surface area of the base surface. Conveyance surface 176 is configured to transport medical waste toward distal-most edge 164 while preventing contact between the medical waste and base surface 172, due to the raised position of the conveyance surface. Therefore, items on tray 160 only contact a very small surface area of the tray, resulting In less frictional resistance between top surface 166 and the items. The relatively low amount of frictional resistance minimizes the effect of friction on items as they roll or slide along landing surface 163.

Rails provided on trays in accordance with the present disclosure can have various shapes, dimensions, and arrangements for minimizing contact between items on the tray and the base surface. Shapes, dimensions, and arrangements of rails can be selected for specific sizes and types of waste, specific sizes and types of containers, and/or other criteria. For example, rails may have specific height to width ratios and/or spacings between rails that are within certain ranges, the ranges being selected based on dimensions of a specific type of waste. Using this criteria, a rail spacing (i.e. the transverse distance between adjacent parallel rails) can be selected to be smaller than a specific article to be discarded. A rail spacing may be selected, for example, to be smaller than the width of a scalpel blade, so that scalpel blades of that type land on top surfaces of the rails, with a very low probability of contacting the base surface. The rail spacing may be selected to be 33% of the width of the scalpel blade, for example. It will be appreciated that smaller or larger percentages can also be used to reduce contact between scalpel blades and the base surface, and different ranges may be desired to address items with other dimensions.

To reduce the surface area that comes in contact with items on the tray, and to facilitate sliding or rolling of items, the top surfaces of rails can have specific geometries that reduce friction and induce rolling. For example, the top edges of rails may be rounded and/or narrowly tapered.

Referring to FIGS. 5 and 5A, rails 174 will be described in additional detail. Each rail 174 has an elongated body 175 that extends parallel to tray axis Y. In addition, each rail 174 has a proximal end 177 that joins to a proximal end of an adjacent rail, forming a parabolic or arc-shaped junction 178. Each junction 178 originates at a point near proximal-most edge 162. Each rail 174 also has a distal end 179 that terminates at distal-most edge 164 of tray 160.

Each rail 174 has a first rail wall 182 and a second rail wall 184 opposite the first rail wall. The majority of first rail walls 182 and second rail walls 184 extend parallel to tray axis Y. Proximal portions of first rail walls 182 and second rail walls 184 extend non-parallel to tray axis Y at points near where the rails converge toward adjacent rails. Each rail 174 includes a rounded top surface 186 that extends between first rail wall 182 and second rail wall 184. Top surfaces 186 are raised above base surface 172, collectively forming conveyance surface 176.

Rails 174 project upwardly from base surface 172 by a height H. Height H can be equal to the height dimension of the first and second rails walls, as in the case where the top surface is flat. Alternatively, height H can be greater than the height dimension of the first and second rails walls, such as where the top surface has an upwardly extending geometry. In the latter case, the top surface can be have various upwardly extending geometries, including but not limited to a pointed geometry, a rounded geometry or any regular or irregular polygonal or curved shape extending above the first and second rail walls. Referring to the present example in FIG. 5A, each rail 174 has a height H that is greater than the height of the first and second rail walls, due to the rounded top surface 186.

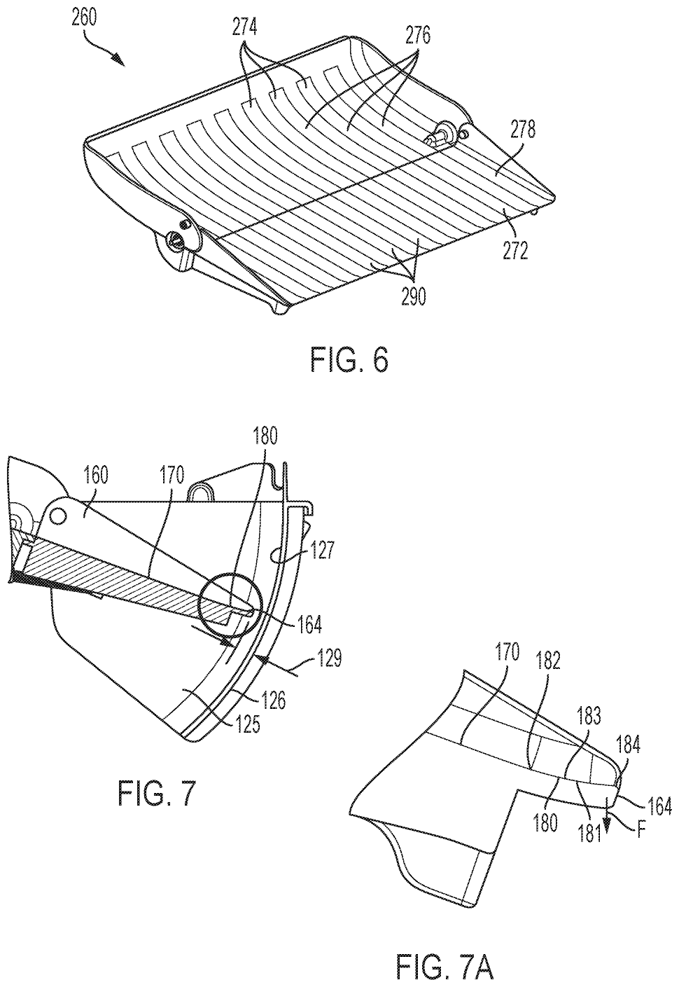

FIG. 6 shows another tray 260 in accordance with the present disclosure. Tray 260 is identical to tray 160 in many respects, but features a plurality of rails 274 that are configured as flat strips 276. Each flat strip 276 has a raised top surface 278 that projects above a base surface 272. Items that are dropped onto tray 260 are supported by top surfaces 278 and suspended above base surface 272. This arrangement decreases the amount of surface area of tray 260 in contact with items dropped onto the tray. Items supported on top surfaces 278 are kept out of contact with base surface 272. In addition, the areas between adjacent strips 276 form channels 290.

The functions of channels in accordance with the present disclosure will now be described in more detail. Trays in accordance with the disclosure can be configured not only to reduce contact between items and the base surface, but can also be designed to address problems associated with liquids. The inventors have found that adding channels and/or other conveyance structures to the tray surface can enhance drainage of liquid from the tray surface after liquid contacts the tray. The conveyance of liquid along a sloped tray surface can be generally thought of in terms of two types of flow: (1) sheet flow and (2) channel flow.

In sheet flow, the liquid is allowed to disperse outwardly in multiple directions on the tray surface. The inventors have found that liquid tends not to pool on flat tray surfaces during sheet flow, but spreads outwardly over a large area. Cohesion forces between liquid molecules can cause the dispersed liquid to divide into small droplets that spread out over the tray surface. Due to their small size, the droplets can attach to the tray surface with enough surface adhesion to resist gravitational force. As a result, the droplets do not drain off of the downwardly sloping surface of the tray, but remain on the tray. This is undesirable, because items dropped onto the tray can adhere to the droplets that remain on the tray. In addition, droplets that remain on the tray can dry over time, forming sticky areas that cause items to adhere to the tray.

In channel flow, liquid is confined to a smaller fixed area on the tray surface, rather than spreading out over a large area. The inventors have observed that channel flow can minimize the formation of small droplets because the confined area of flow causes the liquid volume to remain more concentrated. Gravitational force on the concentrated liquid overcomes the surface adhesion, allowing the liquid to drain off of the tray by gravity. For this reason, the inventors have found that tray surfaces that induce channel flow can remove liquids from the tray surface more effectively than tray surfaces that only induce sheet flow.

Referring back to the example in FIGS. 5 and 5A, rails 174 perform multiple functions that work together to maximize acceleration of items on the tray. In particular, rails 174: (1) provide a small conveyance surface on the tops of the rails that reduces friction between items and the tray surface, (2) direct liquid to the base surface between rails, away from the conveyance surface, (3) promote channel flow of liquid between the rails to quickly remove the liquid from the tray surface, and (4) raise items above the base surface where liquid may reside to keep items out of contact with the liquid.

The first rail walls 182, second rail walls 184, and base surface 172 of tray 160 define a plurality of channels 190. Each channel 190 is configured to promote drainage of liquid from the tray by channel flow. In particular, each channel 190 has a channel width W that extends between adjacent rails 174 and perpendicularly to tray axis Y. Liquid that falls onto tray 160 lands either directly in a channel 190, or indirectly into a channel after first landing on a rail 174 and then dripping down a side of the rail into the channel. The small width W of each channel 190 provides a confined landing area that keeps the liquid more concentrated and captive between two rails 174. As such, the rails 174 minimize the propensity of liquid to spread laterally, or transversely to tray axis Y. This prevents liquid from spreading over a large area and dividing into small droplets that adhere to tray 160. Instead, the concentrated liquid between rails develops enough volume to be drained by gravity toward the distal-most edge 164 of tray where the liquid can drip off the tray. In the event that only a small droplet of liquid lands on tray 160, the droplet is much more likely to land on base surface 172 (because the base surface is much larger than conveyance surface 176) where the droplet cannot come in contact with waste supported above it on the conveyance surface.

Rails and channels in accordance with the disclosure can have various shapes, dimensions and arrangements to facilitate the aforementioned functions. As noted earlier, rails in accordance with the disclosure can be spaced apart by various spacings to provide a desired channel width W. In addition, rails can be spaced uniformly across the tray so that the channel width is constant across the tray. Alternatively, rails can be spaced differently from one rail to the next, creating channels with different widths across the tray. In the present example, rails 174 have alternating spacings such that channel widths W alternate between a first width W₁ and a second width W₂ that is smaller than the first width. Channel widths W can be selected based on the size of items expected to be placed on the tray, and/or based on the tray dimensions, and/or other factors. By way of example, channel widths can be between about ⅛ inch to about 1 inch. Other channel widths outside of this range can also be suitable.

Tray 160 is pivotally mounted in container 100, as described previously. When an item is dropped onto landing surface 163 and rolls or slides past a certain point, the combined center of gravity of the tray and item will move closer to the distal-most edge 164 and away from pivot axis X. This shift of the center of gravity causes tray 160 to pivot out of the equilibrium position and toward a drop position. After the item falls off of tray 160 into receptacle 110, center of gravity C causes the tray to pivot back to the equilibrium position.

Trays in accordance with this disclosure can incorporate features to improve the activation response time and sensitivity of the tray during these pivot motions. For example, trays can include features that enhance the sensitivity and responsiveness of the tray when an item, particularly a light-weight item, is placed on the tray, so as to more quickly pivot the tray and move the item off of the tray. Trays in accordance with this disclosure can also incorporate features that shorten the amount of time it takes for the tray to return from a drop position back to the equilibrium position, so that the tray takes less time to be ready for the next item.

Referring to FIGS. 7 and 7A, acceleration zone 170 includes a change in contour 180 that allows items to impart force to the distal end of tray 160 as items roll past the change in contour. The force imparted by the item to tray 160 increases the tray's momentum as it pivots, thereby increasing the tray's pivot velocity. Increased pivot velocity allows tray 160 to rebound more quickly back to the equilibrium position and receive the next item. Trays in accordance with this disclosure can include various changes of contour to increase the tray's momentum. In the present example, the change in contour 180 is in the form of a ramp 181. Ramp 181 forms a concave curve or bend 183 in the acceleration zone 170. Ramp 181 has a proximal-most end 182 in acceleration zone 170 and a distal-most end 184 that coincides with distal-most edge 164 of tray 160. Ramp 181 is inclined upwardly relative to acceleration zone 170, creating a change in direction that causes the trajectory of rolling items to move around bend 183. As an item moves around bend 183, depicted in FIG. 7A, the item exerts a downward force F on tray 160 as it accelerates. Downward force F increases the downward pivot motion of tray, which is already pivoting in the direction of F as the item rolls toward distal-most edge 164. The additional force F increases the pivot velocity of tray 160 so that the tray moves more quickly through its pivot cycle, and consequently returns more quickly to the equilibrium position.

The manner in which container 100 is used to discard a sharps device will now be described with reference to FIGS. 3 and 4. FIG. 3 shows one example of a sharps in the form of a syringe S. To ready the container 100, the user should set the container down on a flat surface, like surface HS in FIG. 1. Next, the user moves first lock members 130 to their unlocked positions and moves lid 120 to the open position. Once lid 120 is in the open position, tray 160 is in the equilibrium position with landing surface 163 positioned to receive syringe S. The user holds syringe S above mouth 104 and drop area 150, and drops the syringe into the drop area.

Syringe S falls by gravity until it lands on landing surface 163 of tray 160, which smoothly transitions the syringe from a more vertical trajectory to a more horizontal trajectory. This smooth transition prevents bounce back, causing syringe S to remain in contact with and roll along the curved contour of tray 160. As syringe S rolls, the syringe is supported on conveyance surface 176 above base surface 172. This minimizes frictional resistance between syringe S and tray 160. In addition, syringe S is kept out of contact with any moisture or residue that may be present on base surface 172. Syringe S is conveyed by gravity toward distal-most edge 164 of tray in the interior of container 100, as shown in FIG. 4.

Syringe S rolls through acceleration zone 170 and around bend 183 formed by ramp 181. As syringe S accelerates around bend 183, syringe S exerts downward force F on tray 160. Downward force F increases the downward pivot motion of tray, which is already pivoting in the direction of F as syringe S rolls toward distal-most edge 164. Syringe 5 rides up ramp 181 and launches off the distal-most edge 164 of tray. Cowl 125 intercepts syringe S and guides it downwardly beneath tray 160 and into storage area 140. Meanwhile, tray 160 continues its downward momentum, which is increased by downward force F, and rebounds quickly to the equilibrium position to receive the next item.

Although the present disclosure makes reference to specific embodiments, the disclosure is not intended to be limited to the details shown. Rather, various modifications may be made in the details, including but not limited to physical arrangements and combinations of features, without departing from the present disclosure, such modifications being contemplated as part of this disclosure as if expressly described herein.

1. A tray for conveying medical waste from a drop area to a storage area of a sharps collection container, the tray comprising a proximal-most edge, a distal-most edge opposite the proximal-most edge, and an acceleration zone between the proximal-most edge and the distal-most edge, the tray defining a tray axis that extends from the proximal-most edge to the distal-most edge, the acceleration zone comprising a base surface and a plurality of elongated rails projecting above the base surface, the elongated rails collectively forming a conveyance surface above the base surface for transporting medical waste toward the distal-most edge while preventing contact between said medical waste and the base surface,
the acceleration zone comprising an arcuate landing adjacent the proximal-most end, and
the arcuate landing comprising a radius of curvature that increases as the arcuate landing extends toward the distal-most end.

2. (canceled)

3. (canceled)

4. The tray for conveying medical waste of claim 1, further comprising a ramp that terminates at the distal-most end of the tray.

5. The tray for conveying medical waste of claim 4, wherein the ramp is inclined upwardly relative to the acceleration zone.

6. The tray for conveying medical waste of claim 1, wherein the plurality of elongated rails extend parallel to one another.

7. The tray for conveying medical waste of claim 1, wherein each elongated rail comprises a first rail wall and a second rail wall opposite the first rail wall.

8. The tray for conveying medical waste of claim 7, wherein the first rail walls and the second rail walls extend parallel to the tray axis.

9. The tray for conveying medical waste of claim 7, wherein the first rail walls, the second rail walls, and the base surface of the tray define a plurality of channels for conveying liquid toward the distal-most end by channel flow.

10. A tray for conveying medical waste from a drop area to a storage area of a sharps collection container, the tray comprising a proximal-most edge, a distal-most edge opposite the proximal-most edge, and an acceleration zone between the proximal-most edge and the distal-most edge, the tray defining a tray axis that extends from the proximal-most edge to the distal-most edge, the acceleration zone comprising a base surface and a plurality of elongated rails projecting above the base surface, the elongated rails collectively forming a conveyance surface above the base surface for transporting medical waste toward the distal-most edge while preventing contact between said medical waste and the base surface,
wherein each elongated rail comprises a first rail wall and a second rail wall opposite the first rail wall,
wherein the first rail walls, the second rail walls, and the base surface of the tray define a plurality of channels for conveying liquid toward the distal-most end by channel flow, and
wherein each channel has a channel width extending transversely to the tray axis, the channel width being between about ⅛ inch to about 1 inch.

11. The tray for conveying medical waste of claim 1, wherein each elongated rail comprises a top surface raised above the base surface, the top surfaces of the elongated rails collectively forming the conveyance surface.

12. A tray for conveying medical waste from a drop area to a storage area of a sharps collection container, the tray comprising a proximal-most edge, a distal-most edge opposite the proximal-most edge, and an acceleration zone between the proximal-most edge and the distal-most edge, the tray defining a tray axis that extends from the proximal-most edge to the distal-most edge, the acceleration zone comprising a base surface and a plurality of elongated rails projecting above the base surface, the elongated rails collectively forming a conveyance surface above the base surface for transporting medical waste toward the distal-most edge while preventing contact between said medical waste and the base surface,
wherein each elongated rail comprises a first rail wall and a second rail wall opposite the first rail wall,
wherein the first rail walls, the second rail walls, and the base surface of the tray define a plurality of channels for conveying liquid toward the distal-most end by channel flow,
wherein each channel has a channel width extending transversely to the tray axis, the channel width being between about ⅛ inch to about 1 inch,
wherein each elongated rail comprises a top surface raised above the base surface, the top surfaces of the elongated rails collectively forming the conveyance surface and
wherein each top surface is rounded.

13. The tray for conveying medical waste of claim 1, further comprising a first side wall and a second side wall, the first and second side walls projecting above the base surface above the elongated rails.

14. The tray for conveying medical waste of claim 13, wherein the first side wall and the second side wall each comprise a pivot mount for pivotally mounting the tray inside a sharps collection system.

15. A sharps collection system comprising the tray according to claim 1.

16. The sharps collection system of claim 15, further comprising:
a receptacle having a plurality of side walls and a bottom wall; and
a lid attached to the receptacle, the lid being pivotable between an open position and a closed position relative to the receptacle;
wherein the tray is mounted in the receptacle,
wherein the tray and the lid form a mouth between the tray and the lid, the mouth forming a portal through which medical waste can be deposited into the receptacle, and
wherein the arcuate landing is positioned beneath the mouth and faces upwardly toward the lid to receive medical waste and immediately move said medical waste into an inner portion of the receptacle after said medical waste lands on the tray.
